(19) United States
(12) Patent Application Publication
(10) Pub. No.: US 2026/0279557 A1
(43) Pub. Date: Sep. 17, 2026

(54) OUTPATIENT PREDICTION METHOD, OUTPATIENT PREDICTION APPARATUS, AND COMPUTER PROGRAM STORED IN RECORDING MEDIUM TO EXECUTE THE METHOD

(71) Applicant: SAMSUNG LIFE PUBLIC WELFARE FOUNDATION, Seoul (KR)

(72) Inventors: Junghwan MOON, Seoul (KR); Jae Kyun CHOI, Seoul (KR); Seok Doo JEONG, Seoul (KR); So Young YOON, Seoul (KR); Jihyun KIM, Seoul (KR)

(73) Assignee: SAMSUNG LIFE PUBLIC WELFARE FOUNDATION, Seoul (KR)

(21) Appl. No.: 19/672,842

(22) Filed: May 11, 2026

Related U.S. Application Data
(63) Continuation of application No. 18/147,192, filed on Dec. 28, 2022.

(30) Foreign Application Priority Data
Jan. 26, 2022 (KR) 10-2022-0011792

(51) Int. Cl. G16H 40/20; G16H 50/70

(52) U.S. Cl. CPC G16H 40/20; G16H 50/70

(57) ABSTRACT

An outpatient prediction method includes obtaining, by an outpatient prediction apparatus, data on a target to be predicted, obtaining, by the outpatient prediction apparatus, past data including at least one of a number of outpatient clinic units in past, a number of outpatients in past, a number of blood-collecting patients in past, a number of computed tomography (CT)/magnetic resonance imaging (MRI) tests in past, a number of outpatients per outpatient clinic unit in past, a number of blood-collecting patients per outpatient number in past, and a number of CT/MRI tests per outpatient number in past, calculating, by the outpatient prediction apparatus, pattern data based on the past data, and predicting, by the outpatient prediction apparatus, at least one of a number of outpatients in future, a number of blood-collecting patients in future, and a number of CT/MRI tests in future based on the pattern data.

### CROSS-REFERENCE TO RELATED APPLICATION(S)

This application is a continuation of and claims benefit under 35 U.S.C. § 120 to U.S. application Ser. No. 18/147,192, filed Dec. 28, 2022, which is based on and claims priority under 35 U.S.C. § 119 to Korean Patent Application No. 10-2022-0011792, filed on Jan. 26, 2022, in the Korean Intellectual Property Office, the entire content of each of which is incorporated by reference herein.

### BACKGROUND

### 1. Field

The disclosure relates to an outpatient prediction method, an outpatient prediction apparatus, and a computer program stored in a recording medium to execute the method, and more particularly, to an outpatient prediction method, an outpatient prediction apparatus, and a computer program stored in a recording medium to execute the method, in which information related to outpatients is predicted by using past data and data on reserved patients.

### 2. Description of the Related Art

With the recent improvement in quality of life and increased interest in health, the number of people using medical services has gradually increased, and thus, there has been a request for efficient management of reservations in providing medical services such as diagnosis, treatment, and surgery performed in hospitals/clinics. To minimize waiting time for individuals and efficiently manage resources for hospitals/clinics, it has recently become common for hospitals/clinics to manage examination schedules through reservations.

However, outpatients other than reserved patients may visit hospitals/clinics to receive medical services. Blood sampling tests and computed tomography (CT)/magnetic resonance imaging (MRI) tests may be additionally performed depending on conditions of the outpatients. Also, because medical personnel and staff of the hospitals/clinics are assigned regardless the number of outpatients who actually visit or the number of tests that are actually performed, resources of the hospitals/clinics may be insufficient or remain.

### SUMMARY

Provided are an outpatient prediction method, an outpatient prediction apparatus, and a computer program stored in a recording medium to execute the method, in which the number of outpatients in the future, the number of blood-collecting patients in the future, and the number of tests in the future are calculated by predicting information related to outpatients by using past data and data on reserved patients. However, the embodiments are examples, and do not limit the scope of the disclosure.

Additional aspects will be set forth in part in the description which follows and, in part, will be apparent from the description, or may be learned by practice of the presented embodiments.

According to an aspect of the disclosure, an outpatient prediction method includes obtaining, by an outpatient prediction apparatus, data on a target to be predicted, obtaining, by the outpatient prediction apparatus, past data including at least one of a number of outpatient clinic units in past, a number of outpatients in past, a number of blood-collecting patients in past, a number of computed tomography (CT)/magnetic resonance imaging (MRI) tests in past, a number of outpatients per outpatient clinic unit in past, a number of blood-collecting patients per outpatient number in past, and a number of CT/MRI tests per outpatient number in past, calculating, by the outpatient prediction apparatus, pattern data based on the past data, and predicting, by the outpatient prediction apparatus, at least one of a number of outpatients in future, a number of blood-collecting patients in future, and a number of CT/MRI tests in future based on the pattern data.

The predicting may include predicting the number of outpatients in future by using pattern data including at least one of a number of reserved patients in future, a number of outpatient clinic units in past/future, a number of outpatients per outpatient clinic unit in past, a value obtained by multiplying a number of outpatient clinic units in past/future by a number of outpatients per outpatient clinic unit in past, a moving average of a number of outpatients in past, and a moving average of a number of reserved patients in past.

The predicting may include predicting the number of outpatients in future, by using at least one of a model having a number of reserved patients as an input variable, a model having outpatient clinic units and a number of outpatients per outpatient clinic unit as an input variable, a model having a moving average of a number of outpatients as an input variable, and a model having a moving average of a number of reserved patients as an input variable.

The predicting may include predicting the number of outpatients in future by using a model that achieves a lowest mean absolute percentage error (MAPE) from among a model having a number of reserved patients as an input variable, a model having outpatient clinic units and a number of outpatients per outpatient clinic unit as an input variable, a model having a moving average of a number of outpatients as an input variable, and a model having a moving average of a number of reserved patients as an input variable.

A model used to predict the number of outpatients in future may be a model that is a machine-learned through training data having past data as an input and an actual number of outpatients as an output.

The predicting may include calculating the number of blood-collecting patients in future by using the number of outpatients in future and the number of blood-collecting patients per outpatient number, or calculating the CT/MRI tests in future by using the number of outpatients in future and the number of CT/MRI tests per outpatient number.

According to another aspect of the disclosure, an outpatient prediction apparatus includes a non-transitory memory storing one or more computer-readable instructions, and a processor configured to execute the one or more computer-readable instructions stored in the memory to obtain data on a target to be predicted, obtain past data including at least one of a number of outpatient clinic units in past, a number of outpatients in past, a number of blood-collecting patients in past, a number of computed tomography (CT)/magnetic resonance imaging (MRI) tests in past, a number of outpatients per outpatient clinic unit in past, a number of blood-collecting patients per outpatient number in past, and a number of CT/MRI tests per outpatient number in past, calculate pattern data based on the past data, and predict at least one a number of outpatients in future, a number of blood-collecting patients in future, and a number of CT/MRI tests in future based on the pattern data.

The processor may be further configured to predict the number of outpatients in future by using pattern data including at least one of a number of reserved patients in future, a number of outpatient clinic units in past/future, a number of outpatients per outpatient clinic unit in past, a value obtained by multiplying a number of outpatient clinic units in past/future by a number of outpatients per outpatient clinic unit in past, a moving average of a number of outpatients in past, and a moving average of a number of reserved patients in past.

The processor may be further configured to predict the number of outpatients in future, by using at least one of a model having a number of reserved patients as an input variable, a model having outpatient clinic units and a number of outpatients per outpatient clinic unit as an input variable, a model having a moving average of a number of outpatients as an input variable, and a model having a moving average of a number of reserved patients as an input variable.

The processor may be further configured to predict the number of outpatients in future by using a model that achieves a lowest mean absolute percentage error (MAPE) from among a model having a number of reserved patients as an input variable, a model having outpatient clinic units and a number of outpatients per outpatient clinic unit as an input variable, a model having a moving average of a number of outpatients as an input variable, and a model having a moving average of a number of reserved patients as an input variable.

A model used to predict the number of outpatients in future may be a model that is machine-learned through training data having past data as an input and an actual number of outpatients as an output.

The processor may be further configured to calculate the number of blood-collecting patients in future by using the number of outpatients in future and the number of blood-collecting patients per outpatient number, or calculate the number of CT/MRI tests in future by using the number of outpatients in future and the number of CT/MRI tests per outpatient number.

According to another aspect of the disclosure, there is provided a non-transitory computer-readable recording medium storing therein an operating program that causes a computer to execute a process including obtaining data on a target to be predicted, obtaining past data including at least one of a number of outpatient clinic units in past, a number of outpatients in past, a number of blood-collecting patients in past, a number of computed tomography (CT)/magnetic resonance imaging (MRI) tests in past, a number of outpatients per outpatient clinic unit in past, a number of blood-collecting patients per outpatient number in past, and a number of CT/MRI tests per outpatient number in past, calculating pattern data based on the past data, and predicting at least one of a number of outpatients in future, a number of blood-collecting patients in future, and a number of CT/MRI tests in future based on the pattern data.

Other aspects, features, and advantages of the disclosure will become more apparent from the detailed description, the claims, and the drawings.

### BRIEF DESCRIPTION OF THE DRAWINGS

The above and other aspects, features, and advantages of certain embodiments will be more apparent from the following description taken in conjunction with the accompanying drawings, in which:

FIG. 1 is a diagram illustrating a network environment of an outpatient prediction system, according to an embodiment;

FIG. 2 is a diagram for describing a configuration and an operation of an outpatient prediction apparatus, according to an embodiment; FIG. 3 is a block diagram illustrating a memory, according to an embodiment;

FIG. 4 is a flowchart illustrating an outpatient prediction method, according to an embodiment;

FIG. 5 is a diagram illustrating pattern data calculated by an outpatient prediction apparatus;

FIG. 6 is a table including a correlation coefficient between factors calculated according to embodiments;

FIG. 7 is a diagram illustrating how an average absolute error ratio (MAPE, shown as target) of a model changes as a parameter value of the model used for outpatient prediction changes, according to embodiments;

FIG. 8 is a diagram illustrating the number of future outpatients in the future predicted by using a certain training model;

FIG. 9 is a diagram illustrating a method of combining models for predicting the number of outpatients; and

FIG. 10 is a table showing a correlation coefficient between factors used to predict the number of blood-collecting patients by using the outpatient prediction apparatus and the number of blood-collecting patients in the future predicted by using a certain training model, according to embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As the disclosure allows for various changes and numerous embodiments, certain embodiments will be illustrated in the drawings and described in the detailed description. Effects and features of the disclosure, and methods for achieving them will be clarified with reference to embodiments described below in detail with reference to the drawings. However, the disclosure is not limited to the following embodiments and may be embodied in various forms.

Hereinafter, embodiments will be described in detail with reference to the accompanying drawings, wherein the same or corresponding elements are denoted by the same reference numerals throughout and a repeated description thereof is omitted.

Although the terms “first,” “second,” etc. may be used to describe various elements, these elements should not be limited by these terms. These terms are only used to distinguish one element from another. The singular forms “a,” “an,” and “the” are intended to include the plural forms as well, unless the context clearly indicates otherwise. It will be further understood that the terms “comprises” or “comprising” used herein specify the presence of stated features or components, but do not preclude the presence or addition of one or more other features or components.

Sizes of components in the drawings may be exaggerated or contracted for convenience of explanation. For example, because sizes and thicknesses of elements in the drawings are arbitrarily illustrated for convenience of explanation, the disclosure is not limited thereto.

It will be further understood that, when a region, component, unit, block, or module is referred to as being “on” another region, component, unit, block, or module, it may be directly on the other region, component, unit, block, or module or may be indirectly on the other region, component, unit, block, or module with intervening regions, components, units, blocks, or modules therebetween. It will be further understood that, when a region, component, unit, block, or module is referred to as being connected to another region, component, unit, block, or module, it may be directly connected to the other region, component, unit, block, or module or may be indirectly connected to the other region, component, unit, block, or module with intervening regions, components, units, blocks, or modules therebetween.

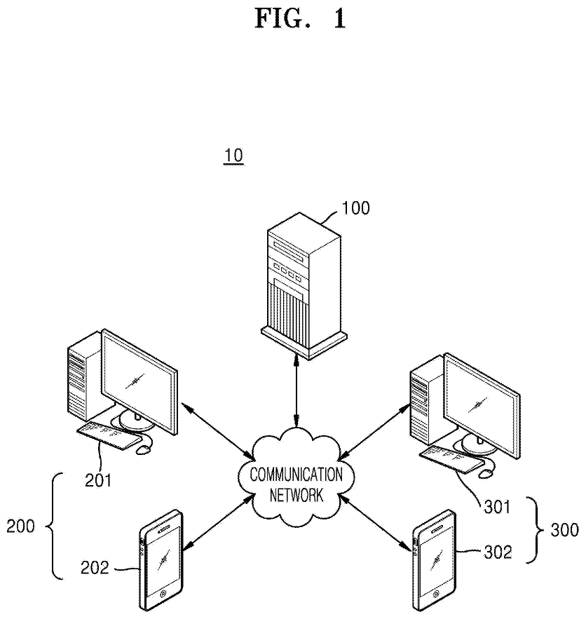

Hereinafter, pattern data refers to data on types and/or rules repeatedly occurring in data. Pattern data may include not only general statistical data (e.g., average value, moving average value, and weighted average value) but also data repeatedly generated between an input variable and an output variable. FIG. 1 is a diagram illustrating a network environment of an outpatient prediction system, according to an embodiment.

An outpatient prediction system 10 may include an outpatient prediction apparatus 100, reservation person terminals 201 and 202 (hereinafter, 200), and staff terminals 301 and 302 (hereinafter, 300).

The outpatient prediction apparatus 100 may obtain reservation data for hospital outpatient treatment from the reservation person terminal 200. The outpatient prediction apparatus 100 may obtain the number of outpatients/blood-collecting patients and the number of computed tomography (CT)/magnetic resonance imaging (MRI) tests by day/time slot/location/department/doctor which are data on tests performed in a hospital from the medical staff terminal 300. Alternatively, the outpatient prediction apparatus 100 may obtain data from a database in which the number of outpatients/blood-collecting patients and the number CT/MRI tests by day/time slot/location/department/doctor which are data on tests performed in a hospital are stored. However, the disclosure is not limited thereto, and the outpatient prediction apparatus 100 may obtain various types of medical data of the hospital. The outpatient prediction system 100 may predict outpatients of one or more hospitals, and may dynamically generate a work schedule of each hospital. The outpatient prediction system 100 may dynamically calculate a work schedule of each hospital by managing the number of outpatients, the number of blood-collecting patients, and the number of CT/MRI tests.

The outpatient prediction apparatus 100 may calculate pattern data related to outpatients based on data on treatment, examination, etc. performed in a hospital, and may predict future data such as the number of outpatients/blood-collecting patients and the number of CT/MRI tests by day/time slot/location/department/doctor based on the pattern data. Also, the outpatient prediction apparatus 100 may generate a work schedule of staff members of the hospital based on the future data. The outpatient prediction apparatus 100 may adjust the number of staff members related to outpatients in response to the number of predicted outpatients. The outpatient prediction apparatus 100 may adjust the number of staff members related to blood sampling in response to the number of predicted blood-collecting patients. The outpatient prediction apparatus 100 may adjust the number of staff members related to CT/MRI tests in response to the number of predicted CT tests or the number of predicted MRI tests. The outpatient prediction apparatus 100 is not limited thereto and may generate various data on hospital staff members.

A reservation person may input reservation data for outpatient treatment through at least one reservation person terminal 200. The reservation person terminal 200 may be connected to a communication network to access the outpatient prediction apparatus 100, and may input reservation data for outpatient treatment. The reservation person terminal 200 may be a desktop or a smartphone as shown in FIG. 1, but the disclosure is not limited thereto and the reservation person terminal 200 refers to a computing device including a processor.

A hospital staff member may input data on outpatients, data related to blood sampling, and data on the number of tests through at least one staff terminal 300. The staff terminal 300 may be a desktop or a smartphone as shown in FIG. 1, but the disclosure is not limited thereto and the staff terminal 300 refers to a computing device including a processor.

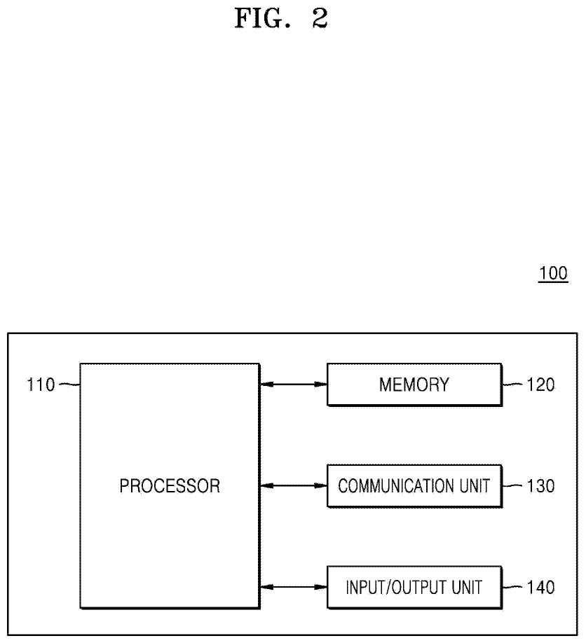

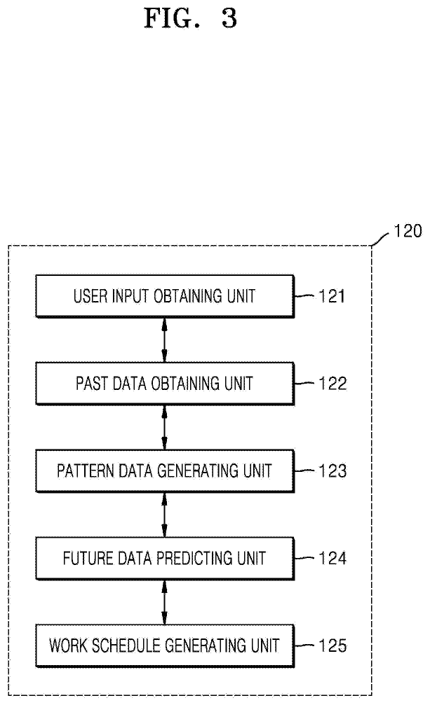

FIG. 2 is a diagram for describing a configuration and an operation of an outpatient prediction apparatus, according to an embodiment. FIG. 3 is a block diagram illustrating a memory, according to an embodiment.

First, referring to FIG. 2, the outpatient prediction apparatus 100 according to an embodiment may include a processor 110, a memory 120, a communication unit 130, and an input/output unit 140. However, the disclosure is not limited thereto, the outpatient prediction apparatus 100 may include more elements or some elements may be omitted. Some elements of the outpatient prediction apparatus 100 may be separated into a plurality of devices, or a plurality of elements may be combined into one device.

The processor 110 may obtain data on outpatients stored in the memory. The processor 110 may predict the number of outpatients, blood-collecting patients, and CT/MRI tests in the future based on data related to outpatients, data related to blood sampling, and data related to CT/MRI tests by day, time slot, location, department, or doctor. The processor 110 may generate a schedule of staff members of a hospital by considering the predicted number of outpatients, blood-collecting patients, and CT/MRI tests in the future. Staff members corresponding to the predicted number of outpatients, blood-collecting patients, and CT/MRI tests in the future may be assigned. A minimum value may be set for each of the staff members corresponding to the predicted number of outpatients, blood-collecting patients, and CT/MRI test in the future, and a schedule may be generated so that staff members corresponding to the minimum value or more are assigned in each unit time.

The memory 120 is a computer-readable recording medium, and may include a random-access memory (RAM), a read-only memory (ROM), and a permanent mass storage device such as a disk drive. Also, program code and a prediction model for controlling the outpatient prediction apparatus 100 may be temporarily or permanently stored in the memory 120. For example, the memory 120 may store data on outpatients, data on reserved patients, data on blood sampling, and/or data on CT/MRI tests.

The communication unit 130 may provide a function of communicating with an external server, a terminal, or a database through a network. For example, a request generated by the processor 110 of the outpatient prediction apparatus 100 according to program code stored in a recording device such as the memory 120 may be transmitted to the external server through the network under the control of the communication unit 130. Conversely, a control signal, a command, content, or a file provided under the control of a processor of the external server may be received by the outpatient prediction apparatus 100, through the communication unit 130 through the network. For example, a control signal or a command of the external server received through the communication unit 130 may be transmitted to the processor 110 or the memory 120.

A communication method is not limited, and may include not only a communication method using a communication network (e.g., a mobile communication network, wired Internet, wireless Internet, or a broadcasting network) that may be included in the network, but also short-range wireless communication between devices. For example, the network may include at least one of a personal area network (PAN), a local area network (LAN), a campus area network (CAN), a metropolitan area network (MAN), a wide area network (WAN), a broadband network (BBN), and the Internet. Also, the network may include, but is not limited to, at least one of a network topology including a bus network, a star network, a ring network, a mesh network, a star-bus network, and a tree or hierarchical network.

Also, the communication unit 130 may communicate with the external server through the network. The communication method is not limited, but the network may be a short-range wireless communication network. For example, the network may be a Bluetooth, BLE, and Wi-Fi communication network.

Also, the outpatient prediction apparatus 100 according to the disclosure may include the input/output unit 140. The input/output unit 140 may be a means for interfacing with an input/output device. For example, the input device may include a device such as a keyboard or a mouse, and the output device may include a device such as a display for displaying a communication session of an application. In another example, the input/output unit 140 may be a means for interfacing with a device in which input and output functions are integrated such as a touchscreen. In a specific example, when the processor 110 of the outpatient prediction apparatus 100 processes a command of a program loaded or stored in the memory 120, a service screen or content may be displayed on a display through the input/output unit 140.

Also, in other embodiments, the outpatient prediction apparatus 100 may include more elements than those illustrated in FIG. 2. For example, the outpatient prediction apparatus 100 may include at least some of the above input/output devices, or may further include other elements such as a battery and a charging device for supplying power to internal elements, various sensors, and a database.

An internal configuration of the memory 120 of the outpatient prediction apparatus 100 according to an embodiment will be described in detail with reference to FIG. 3. For better understanding, the following will be described assuming that the memory 120 is the memory 110 of the outpatient prediction apparatus 100 illustrated in FIG. 2.

The memory 120 of the outpatient prediction apparatus 100 according to an embodiment includes a user input obtaining unit 121, a past data obtaining unit 122, a pattern data generating unit 123, a future data predicting unit 124, and a work schedule generating unit 125.

The user input obtaining unit 121 may obtain data on a target to be predicted. The data on the target may include a period to be predicted, a type of data to be predicted, a treatment depart to be predicted, a doctor to be predicted, and an examination room to be predicted, but the disclosure is not limited thereto and the data on the target may include values that specify the target to be predicted.

The past data obtaining unit 122 may obtain past data including the number of outpatient clinic units in the past, the number of outpatients in the past, the number of blood-collecting patients in the past, the number of CT/MRI tests in the past, the number of outpatients per outpatient clinic unit in the past, the number of blood-collecting patients per outpatient number in the past, and the number of CT/MRI tests per outpatient number in the past. The past data obtaining unit 122 may obtain such data as time-series data by date, working day, day of the week, and department. The past data obtaining unit 122 may classify and obtain such data based on each hospital, department, doctor, and examination room.

The pattern data generating unit 123 may calculate data obtained by the past data obtaining unit 122 as pattern data by time slot, department, and doctor. Additionally, the pattern data generating unit 123 may obtain the number of reserved patients in the past or the number of reserved patients in the future. For example, the pattern data may include the number of reserved patients, a moving average value of the number of reserved patients, a moving average value of the number of outpatients, the number of outpatient clinic units, the number of outpatients per outpatient clinic unit, a correlation coefficient between the number of outpatients in the past and the number of reserved patients, a correlation coefficient between the number of outpatients in the past and a moving average value of the number of reserved patients, a correlation coefficient between the number of outpatients in the past and a moving average value of the number of outpatients, a correlation coefficient between the number of outpatients in the past and the number of outpatient clinic units, and a correlation coefficient between the number of outpatients in the past and the number of outpatients per outpatient clinic unit.

The pattern data generating unit 123 may calculate the number of outpatients per outpatient clinic unit, the number of blood-collecting patients per outpatient clinic unit, and the number of CT/MRI tests per outpatient clinic unit. A outpatient clinic unit may be a unit in which treatment divided into a department and a doctor is performed.

The future data predicting unit 124 may predict the number of outpatients in the future, the number of blood-collecting patients in the future, and the number of CT/MRI tests in the future based on pattern data.

The future data predicting unit 124 may calculate the number of outpatients in the future, the number of blood-collecting patients in the future, and the number of CT/MRI tests in the future by using a model that is trained with past pattern data or a correlation coefficient and data generated in the future (future in terms of past data but past in terms of present), through the past data. In this case, the trained model may be generated by using various methodologies of machine learning, and parameters may be optimized by using a boosting-based regress model. The trained model may be optimized by using a method such as particle swarm optimization, meta heuristic algorithm, or Bayesian optimization. The trained model may be generated in a determined evaluation period and a determined validation period. The trained model may be tuned based on validation data. The trained model may be generated through the outpatient prediction apparatus 100 or an external server. A plurality of trained models may be generated separately according to a target to be predicted.

The future data predicting unit 124 may calculate, by using past data, a correlation coefficient between the number of outpatients and past data of the number of reserved patients, the number of outpatients, the number of outpatient clinic units, the number of outpatients per outpatient clinic unit, a outpatient clinic unit*the number outpatients per outpatient clinic unit, a moving average of the number of outpatients, and a moving average of the number of reserved patients. The future data predicting unit 124 may calculate the number of outpatient clinic units in the future based on the correlation coefficient. The future data predicting unit 124 may calculate the number of outpatients in the future by multiplying the number of outpatient clinic units in the future by the number of outpatients per outpatient clinic unit. The future data predicting unit 124 may calculate the number of tests, the number of blood sampling, and the number of other related patients in the future, by using the number of outpatients in the future. In this case, the future data predicting unit 124 may calculate the number of outpatients in the future by using pattern data of the number of tests per outpatient number in the past or the number of blood sampling per outpatient number in the past.

The future data predicting unit 124 may create pattern data of the number of outpatients by working data, date, and department from the number of outpatients in the past, and may calculate the number of outpatients in the future based on the pattern data. In this case, the number of outpatients in the future may be calculated by combining a maximum value, a minimum value, a median value, and an average of the number of outpatients in the future derived by various prediction models or by selecting one of the maximum value, the minimum value, the median value, and the average of the derived number of outpatients in the future. The number of outpatients in the future may correspond to a model trained by using various input variables such as a reserved patient number model, a outpatient clinic unit model, an outpatient number moving average model, or a reserved patient number moving average model. The reserved patient number model may be a model trained to calculate the number of reserved patients in the future from past data and output the number of outpatients in the future by using the number of reserved patients in the future. The outpatient clinic unit model may be a model trained to calculate the number of outpatient clinic units in the future from past data and output the number of outpatients in the future by using the number of outpatient clinic units in the future.

The future data predicting unit 124 may create pattern data of the number of blood-collecting patients by working day, date, and department from the number of blood-collecting patients in the past, and may calculate the number of blood-collecting patients in the future based on the pattern data. The future data predicting unit 124 may create pattern data of the number of CT/MRI tests by working day, date, and department from the number of CT/MRI tests in the past, and may calculate the number of CT/MRI tests in the future based on the pattern data.

The work schedule generating unit 125 may generate a work schedule of staff of a hospital based on at least one of the number of outpatients in the future, the number of blood-collecting patients in the future, and the number of CT/MRI tests in the future generated by the future data predicting unit 124. The work schedule may include the number of staff members assigned to each examination room and a work timetable of each staff member.

The work schedule generating unit 125 may calculate the number of outpatients by day/time slot/department/doctor and may generate a first work schedule of hospital staff members related to outpatients.

The work schedule generating unit 125 may calculate the number of blood-collecting patients by day/time slot/location and may generate a second work schedule related to hospital staff members related to blood sampling. The number of blood-collecting patients by day/time slot/location may be calculated based on the number of blood-collecting patients by department and/or the number of blood-collecting patients by doctor. That is, the work schedule generating unit 125 may also calculate the number of blood-collecting patients by department and/or the number of blood-collecting patients by doctor based on pattern data on the number of blood-collecting patients.

The work schedule generating unit 125 may calculate the number of CT/MRI tests by day/time slot/location and may generate a third work schedule of hospital staff members related to CT/MRI. The number of CT/MRI tests by day/time slot/location in the future may be calculated based on the number of CT/MRI tests by department in the future and/or the number of CT/MRI tests by doctor in the future.

The processor 110 may control the outpatient prediction apparatus 100 to execute the memory 120 of FIG. 3 to perform operations S110 to S150 included in an outpatient prediction method.

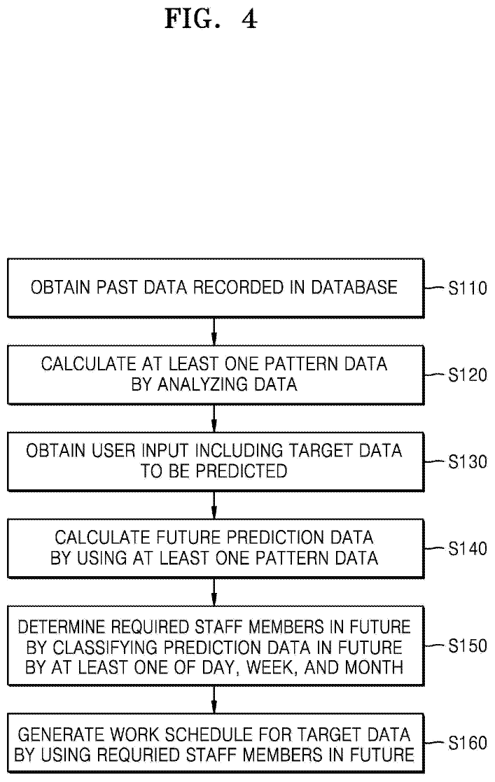

FIG. 4 is a flowchart illustrating an outpatient prediction method, according to an embodiment.

In operation S110, the outpatient prediction apparatus 100 may obtain past data. The past data may include the number of outpatients, the number of outpatient clinic units, the number of blood-collecting patients, and the number of CT/MRI tests classified by date, department, working day, and day of the week. The outpatient prediction apparatus 100 may classify and obtain such data based on each hospital, department, doctor, and examination room.

In operation S120, the outpatient predicting apparatus 100 may calculate at least one pattern data by analyzing the past data. The outpatient prediction apparatus 100 may calculate pattern data related to the number of outpatients, the number of blood-collecting patients, the number of outpatient clinic units, and the number of CT/MRI tests by analyzing the past data. The outpatient prediction apparatus 100 may calculate pattern data including the number of reserved patients, a moving average value of the number of reserved patients, a moving average value of the number of outpatients, the number of outpatient clinic units, and the number of outpatients per outpatient clinic unit as factors related to the number of outpatients. The outpatient prediction apparatus 100 may calculate a correlation coefficient between the number of outpatients in the past and the number of reserved patients, a correlation coefficient between the number of outpatients in the past and the moving average value of the number of reserved patients, a correlation coefficient between the number of outpatients in the past and the moving average value of the number of outpatients, a correlation coefficient between the number of outpatients in the past and the number of outpatient clinic units, and a correlation coefficient between the number of outpatients in the past and the number of outpatients per outpatient clinic unit. In operation S130, the outpatient prediction apparatus 100 may obtain a user input including target data to be predicted. The outpatient prediction apparatus 100 may obtain data on a target to be predicted. The data on the target may include a period to be predicted, a type of data to be predicted, a department to be predicted, a doctor to be predicted, and an examination room to be predicted, but the disclosure is not limited thereto and the data on the target may include values that specify the target to be predicted.

In operation S140, the outpatient prediction apparatus 100 may calculate future prediction data corresponding to the user input by using the at least one pattern data. The outpatient prediction apparatus 100 may create pattern data of the number of outpatients, by using past data classified by working day, date, day of the week, and department from the number of outpatients in the past. Based on the pattern data, the final number of outpatients in the future may be calculated by combining a maximum value, a minimum value, a median value, and an average of the number of outpatients in the future derived by any of various prediction models. In this case, the prediction model may be one of a reserved patient number model, a outpatient clinic unit model, an outpatient number moving average model, and a reserved patient number moving average model. The reserved patient number model may be a model trained to calculate the number of reserved patients in the future from past data, and output the number of outpatients in the future by using the number of reserved patients in the future. The outpatient clinic unit model may be a model trained to calculate the number of outpatient clinic units in the future from past data, and output the number of outpatients in the future by using the number of outpatient clinic units in the future. In more detail, the outpatient prediction apparatus 100 may calculate the number of outpatient clinic units in the future based on pattern data, and may calculate the number of outpatients in the future by multiplying the number of outpatient clinic units in the future by the number outpatients per outpatient clinic unit.

The outpatient prediction apparatus 100 may create pattern data of the number of blood-collecting patients in the order of working day, department, and time from the number of blood-collecting patients in the past, and may calculate the number of blood-collecting patients in the future based on the pattern data. In more detail, the outpatient prediction apparatus 100 may calculate the number of blood-collecting patients by multiplying the number of outpatients by the number of blood-collecting patients per outpatient number. The outpatient prediction apparatus 100 may calculate the number of blood-collecting patients in the future by multiplying the number of outpatients predicted in the above method by the number of blood-collecting patients per outpatient number included in the pattern data. The outpatient prediction apparatus 100 may create pattern data of the number of CT/MRI tests in the order of working day, day of the week, department, and date from the number of CT/MRI tests in the past, and may calculate the number of CT/MRI tests in the future based on the pattern data. In more detail, the outpatient prediction apparatus 100 may calculate the number of CT/MRI tests in the future by multiplying the number of outpatients which is predicted by the number of CT/MRI tests per outpatient number corresponding to the future included in the pattern data.

In operation S150, the outpatient prediction apparatus 100 may determine required staff members in the future by classifying the prediction data in the future by at least one determined time unit, for example, day, week, or month. The outpatient prediction apparatus 100 may generate a work schedule of hospital staff members based on at least one of the number of outpatients in the future, the number of blood-collecting patients in the future, and the number of CT/MRI tests in the future.

In operation S160, the outpatient prediction apparatus 100 may generate a work schedule for target data by using the required staff members in the future. The outpatient prediction apparatus 100 may generate a first work schedule of hospital staff members related to outpatients by calculating the number of outpatients by day/time slot/department/doctor. The outpatient prediction apparatus 100 may generate a second work schedule of hospital staff members related to blood sampling by calculating the number of blood-collecting patients by day/time slot/location. The outpatient prediction apparatus 100 may generate a third work schedule of hospital staff members related to CT/MRI by calculating the number of CT/MRI tests by day/time slot/location.

The outpatient prediction apparatus 100 may generate a final work schedule of hospital staff members based on the first to third work schedules.

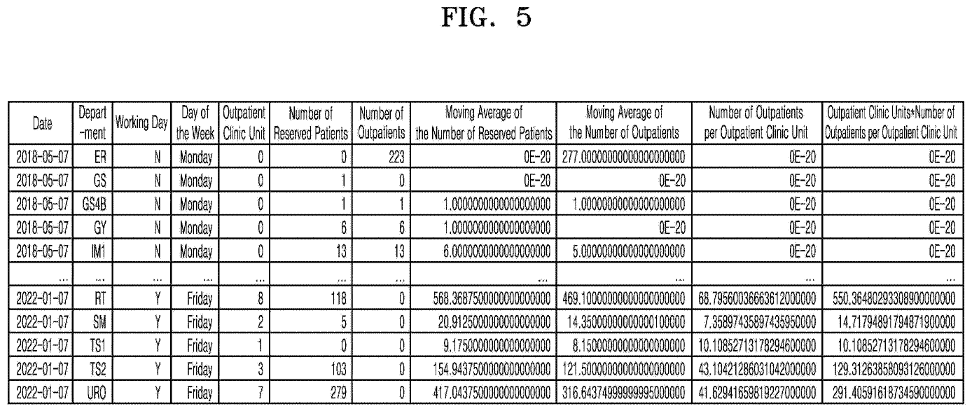

FIG. 5 is a diagram illustrating pattern data calculated by an outpatient prediction apparatus.

As shown in FIG. 5, past data may be standardized by date. The past data may be classified by department or doctor, and may include the number of reserved patients and the number of outpatients. Pattern data such as a moving average of the number of reserved patients, a moving average of the number of outpatients, the number of outpatients per outpatient clinic unit, and a value obtained by multiplying the number of outpatient clinic units by the number of outpatients per outpatient clinic unit may be calculated based on the past data by department or doctor.

The outpatient prediction apparatus 100 may standardize a outpatient clinic unit, the number of reserved patients, and the number of outpatients by ‘date, department, working day, and day of the week’, and may calculate a moving average value of the number of outpatients per outpatient clinic unit. The number of outpatients may be calculated by multiplying the number of outpatient clinic units by the number of outpatients per outpatient clinic unit.

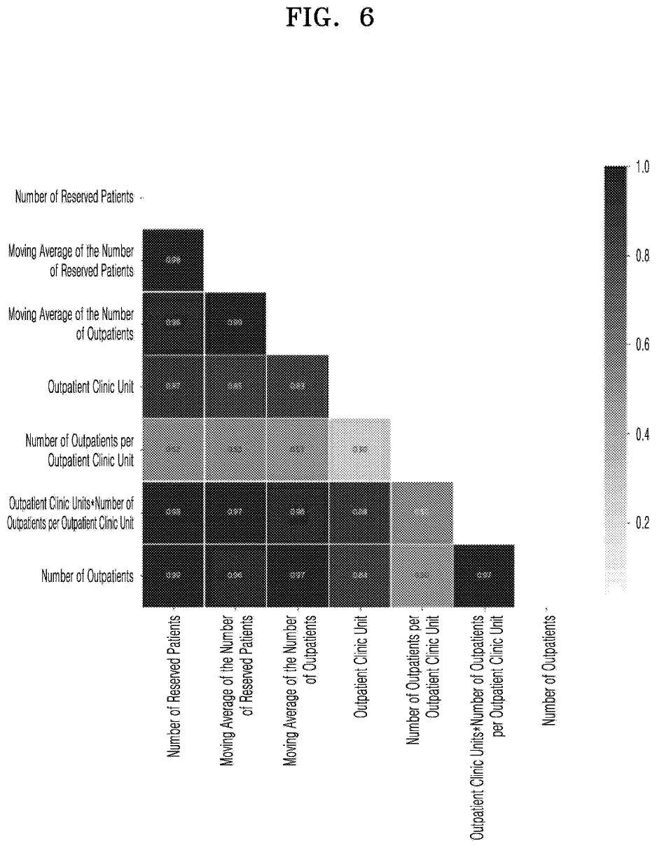

FIG. 6 is a table including a correlation coefficient between factors calculated according to embodiments.

The outpatient prediction apparatus 100 may visually show correlation coefficients as shown in FIG. 6. As a correlation coefficient increases, it may be expressed in a darker color.

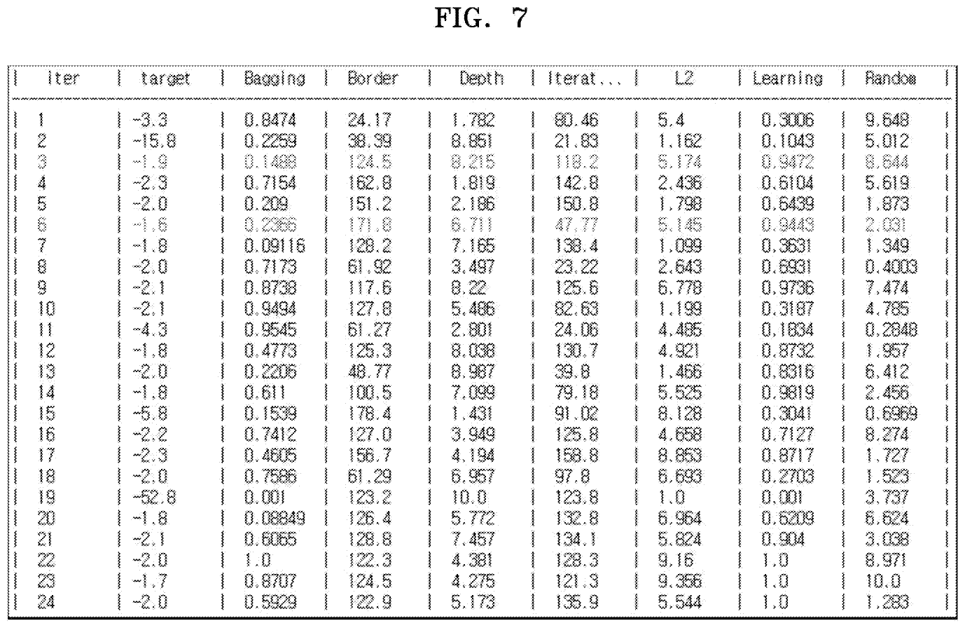

A correlation coefficient between the number of reserved patients and a moving average value of the number of reserved patients may be 0.98, a correlation coefficient between the number of reserved patients and a moving average value of the number of outpatients may be 0.96, a correlation coefficient between the number of reserved patients and the number of outpatient clinic units may be 0.87, a correlation coefficient between the number of reserved patients and the number of outpatients per outpatient clinic unit may be 0.52, a correlation coefficient between the number of reserved patients and the number of outpatients per outpatient clinic unit*outpatient clinic unit may be 0.98, and a correlation coefficient between the number of reserved patients and the number of outpatients may be 0.99. Although a correlation coefficient between the number of reserved patients and the number of outpatients may be a highest value and a correlation coefficient between the number of reserved patients and the number of outpatients per outpatient clinic unit may be a lowest value, the disclosure is not limited thereto and a value calculated by the outpatient prediction apparatus 100 may be borrowed. FIG. 7 is a diagram illustrating parameters of a model used for outpatient prediction, according to embodiments.

The outpatient prediction apparatus 100 may utilize an optimization algorithm such as particle swarm optimization, meta heuristic algorithm, or Bayesian optimization, and may optimize parameters by using a boosting-based regression model (e.g., categorical boosting (CatBoost)) that has a lowest mean absolute percentage error (MAPE) of validation period data. As shown in FIG. 7, the outpatient prediction apparatus 100 may predict the number of outpatients and the number of blood-collecting patients in the future by adjusting values of Bagging, border, depth, L2, learning, and random, and may predict the number of outpatients and the number of blood-collecting patients in the future with parameters of a model having a highest target value. Bagging refers to bagging temperature and border refers to the number of divisions for a numerical feature. Depth refers to a depth of a tree used by the model. Iterations refers to the number of iterations during training, and L2 refers to the number of attempts to find a best value. Random refers to the number of times the order of input data is randomly adjusted, and may refer to parameters defined in CatBoost API. As shown in FIG. 7, the outpatient prediction apparatus 100 may perform a test through a combination of 24 parameters, and may determine a combination of parameters having a highest target (a combination of parameters 6).

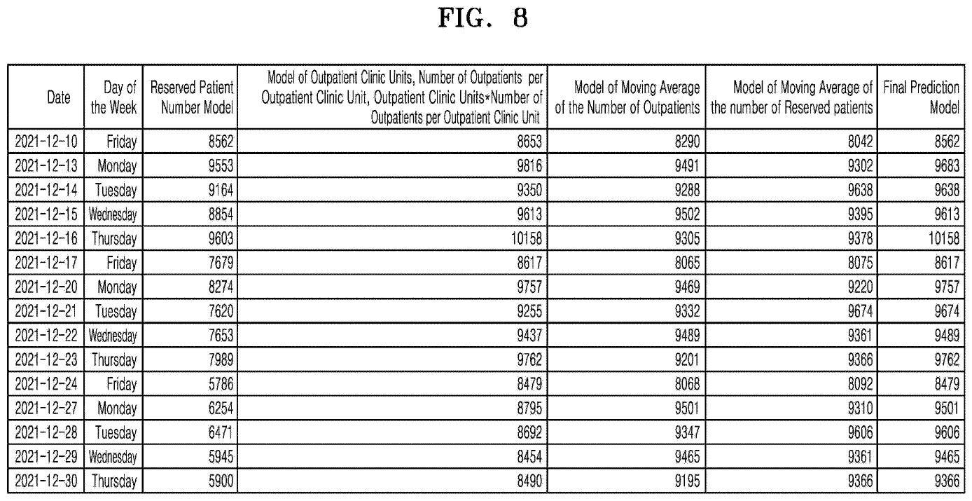

FIG. 8 is a diagram illustrating the number of outpatients in the future predicted by using a certain training model.

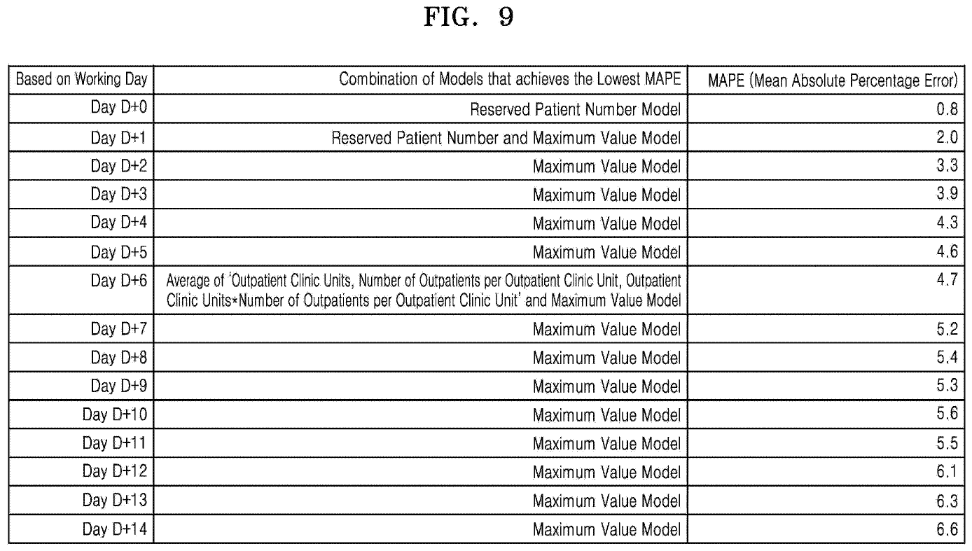

As shown in FIG. 8, the number of outpatients may be predicted by a model having the number of reserved patients as an input variable, a model having outpatient clinic units and the number of outpatients per outpatient clinic unit as an input variable, a model having a moving average of the number of outpatients as an input variable, and a model having a moving average of the number of reserved patients as an input variable. The number of outpatients may be predicted by date. As shown in FIG. 8, 8562 patients may be predicted by the model having the number of reserved patients as an input variable, 8653 patients may be predicted by the model having outpatient clinic units and the number of outpatients per outpatient clinic unit as an input variable, and 8042 patients may be predicted by the model having a moving average of the number of outpatients as an input variable. Also, a final value may be calculated by combining values obtained by the model having the number of reserved patients as an input variable which is a model with a lowest error rate, from among the model having the number of reserved patients as an input variable, the model having outpatient clinic units and the number of outpatients per outpatient clinic unit as an input variable, the model having a moving average of the number of outpatients as an input variable, and the model having a moving average of the number of reserved patients as an input variable. A model with a lowest error rate may vary by date as shown in FIG. 9. On day D+0, 8562 patients of the reserved patient number model with a lowest error rate may be determined, and on day D+1, 9683 patients that corresponds to an average value between a value of the reserved patient number model and a maximum value among calculated values may be determined. FIG. 9 is a diagram illustrating a method of combining models for predicting the number of outpatients.

The outpatient prediction apparatus 100 may select a model that achieves a lowest mean absolute percentage error (MAPE) from among a model having the number of reserved patients as an input variable, a model having outpatient clinic units and the number of outpatients per outpatient clinic unit as an input variable, a model having a moving average of the number of outpatients as an input variable, and a model having a moving average of the number of reserved patients as an input variable. In this case, the outpatient prediction apparatus 100 may select with a lowest MAPE for each date for the future based on a current time point. As shown in FIG. 9, to predict the number of outpatients on day D+1, a reserved patient number and maximum value model may be a model with a lowest MAPE. The outpatient prediction apparatus 100 may determine a model with a lowest MAPE for each date in this way, and may predict the number of outpatients of the date by using the model with the lowest MAPE. A MAPE of each model may be calculated as ‘|forecast value?actual value|/forecast value’, but the disclosure may not be limited and a mean absolute error (MAE), a root mean square error (RMSE), etc. may be applied to calculate an error of a model. As shown in FIG. 9, a value calculated by the reserved patient number model may be output as the number of outpatients in the future on day D+0, an average value between a value of the reserved patient number model and a value of the maximum value model may be output as the number of outpatients in the future on day D+1, and a value of the maximum value model may be output as the number of outpatients in the future on day D+2. The maximum value model means that a maximum value among calculated values is determined as an output value.

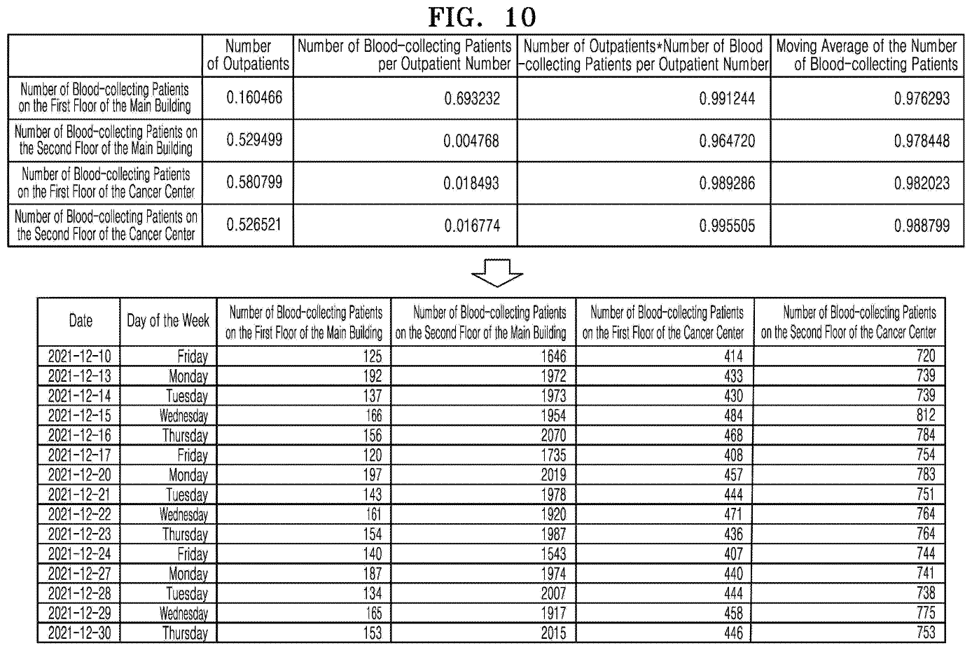

FIG. 10 is a table showing correlation coefficients calculated by the outpatient prediction apparatus 100, according to embodiments.

The outpatient prediction apparatus 100 may predict the number of blood-collecting patients on the first floor of the main building, the number of blood-collecting patients on the second floor of the main building, the number of blood-collecting patients on the first floor of the cancer center, and the number of blood-collecting patients on the second floor of the cancer center. The outpatient prediction apparatus 100 may calculate the number of blood-collecting patients in the future by multiplying the number of outpatients by the number of blood-collecting patients per outpatient number in the future. The number of blood-collecting patients per outpatient number in the future enables to calculate a correlation coefficient between the number of outpatients and the number of blood-collecting patients, a correlation coefficient between the number of blood-collecting patients per outpatient number and the number of blood-collecting patients, a correlation coefficient between the number of blood-collecting patients per outpatient number X the number of outpatients and the number of blood-collecting patients, and a correlation coefficient between a moving average value of the number of blood-collecting patients and the number of blood-collecting patients, calculated through past data. The number of blood-collecting patients in the future may be calculated based on the correlation coefficients. As shown in FIG. 10, the outpatient prediction apparatus 100 may predict the number of blood-collecting patients on the first floor of the main building as 125, 192, 137, . . . by date. The outpatient prediction apparatus 100 may predict the number of blood-collecting patients on the second floor of the main building as 1646, 1972, 1973, 2070, . . . by date. More staff members may be assigned on 2021 Dec. 13 when the number of blood-collecting patients on the first floor of the main building is 192, and fewer staff members may be assigned to the blood sampling room on the first floor of the main building on 2021 Dec. 10 when the number of blood-collecting patients is 125.

The outpatient prediction apparatus 100 may predict the number of blood-collecting patients for each blood sampling room and floor, and may adjust the number of staff members working in each blood sampling room or floor. A device and/or system described herein may be implemented using hardware components, software components, or a combination thereof. A device and an element described in embodiments may be implemented using one or more general-purpose or special purpose computers, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. A processing device may run an operating system (OS) and one or more software applications that run on the OS. Also, the processing device may access, store, manipulate, process, and create data in response to execution of software. For easy understanding, one processing device is used, but it will be understood by one of ordinary skill in the art that a processing device may include multiple processing elements and/or multiple types of processing elements. For example, the processing device may include multiple processors or a processor and a controller. In addition, other processing configurations, such as parallel processors, are possible.

Software may include a computer program, code, instructions, or a combination of one or more of these, to independently or collectively instruct or configure a processing device to operate as desired. Software and/or data may be embodied permanently or temporarily in any type of machine, component, physical or virtual equipment, computer storage medium or device, or a transmitted signal wave, to provide instructions or data to or to be interpreted by a processing device. Software may also be distributed over network coupled computer systems so that the software is stored and executed in a distributed fashion. Software and data may be stored in one or more computer-readable recording media.

A method according to an embodiment may be embodied as program commands executable by various computer means and may be recorded on a computer-readable recording medium. The computer-readable recording medium may include program commands, data files, data structures, and the like separately or in combinations. The program commands recorded on the computer-readable recording medium may be specially designed and configured for embodiments or may be well-known to and be usable by one of ordinary skill in the art of computer software. Examples of the computer-readable recording medium include a magnetic medium such as a hard disk, a floppy disk, or a magnetic tape, an optical medium such as a compact disc read-only memory (CD-ROM) or a digital versatile disk (DVD), a magneto-optical medium such as a floptical disk, and a hardware device specially configured to store and execute program commands such as a ROM, a RAM, or a flash memory. Examples of the program commands include advanced language code that may be executed by a computer by using an interpreter or the like as well as machine language code made by a compiler. The described hardware device may be configured to operate as one or more software modules in order to perform an operation of an embodiment, and vice versa.

According to the one or more embodiments, there may be provided an outpatient prediction method, an outpatient prediction apparatus, and a computer program stored in a recording medium to execute the method, in which a hospital work schedule may be efficiently generated by predicting information related to outpatients by using past data and data on reserved patients. However, the scope of the disclosure is not limited by these effects.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An outpatient prediction apparatus comprising:
a processor; and
a memory storing instructions that, when executed by the processor, cause the processor to:
obtain past data including a number of outpatient clinic units in past, a number of outpatients in past, a number of blood-collecting patients in past, a number of computed tomography (CT)/magnetic resonance imaging (MRI) tests in past, a number of reserved patients in past, and a number of outpatients per outpatient clinic unit in past;
calculate pattern data based on the past data by
(i) calculating a correlation coefficient between the number of outpatients in past and the number of outpatient clinic units in past; and
(ii) generating a composite input variable by multiplying the number of outpatient clinic units in past by a number of outpatients per outpatient clinic unit in past;
generate input variables including the pattern data, a moving average of the number of outpatients in past, and a moving average of the number of reserved patients in past;
train a plurality of machine-learned prediction models using training data derived from the past data and the input variables, and
and optimize parameters of the plurality of machine-learned prediction models;
evaluate prediction errors of the plurality of machine-learned prediction models by calculating a mean absolute percentage error (MAPE) and select a prediction model having a lowest MAPE;
predict a number of outpatients in future using the selected prediction model;
calculate a number of blood-collecting patients in future based on the predicted number of outpatients in future and a number of blood-collecting patients per outpatient number;
calculate a number of CT/MRI tests in future based on the predicted number of outpatients in future and a number of CT/MRI tests per outpatient number; and
generate a work schedule of staff members based on the predicted number of outpatients in future, the predicted number of blood-collecting patients in future, and the predicted number of CT/MRI tests in future, and output the work schedule on a service screen via an input/output unit to adjust a number of staff members assigned to each examination room.

2. The apparatus of claim 1, wherein the input variables further include the number of reserved patients in past.

3. The apparatus of claim 1, wherein the input variables further include the number of outpatient clinic units in past.

4. The apparatus of claim 1, wherein the input variables further include a value obtained by multiplying the number of outpatient clinic units in past or future by the number of outpatients per outpatient clinic unit in past.

5. The apparatus of claim 1, wherein the past data is time-series data classified by at least one of date, department, working day, and day of the week.

6. The apparatus of claim 1, wherein the plurality of machine-learned prediction models comprise a reserved-patient model, an outpatient-clinic-unit model, an outpatient moving-average model, and a reserved-patient moving-average model.

7. The apparatus of claim 1, wherein the plurality of machine-learned prediction models are trained using training data having the past data as an input and an actual number of outpatients as an output.

8. The apparatus of claim 1, wherein parameters of the plurality of machine-learned prediction models are optimized using a boosting-based regression model.

9. The apparatus of claim 8, wherein the boosting-based regression model comprises a categorical boosting model.

10. The apparatus of claim 1, wherein parameters of the plurality of machine-learned prediction models are optimized using particle swarm optimization.

11. The apparatus of claim 1, wherein parameters of the plurality of machine-learned prediction models are optimized using a meta heuristic algorithm.

12. The apparatus of claim 1, wherein parameters of the plurality of machine-learned prediction models are optimized using Bayesian optimization.

13. The apparatus of claim 1, wherein the mean absolute percentage error (MAPE) is used as a technical metric to evaluate and rank a prediction performance of the plurality of machine-learned prediction models.

14. The apparatus of claim 1, wherein the predicted number of blood-collecting patients in future is calculated by multiplying the predicted number of outpatients in future by the number of blood-collecting patients per outpatient number included in the pattern data.

15. The apparatus of claim 1, wherein the predicted number of CT/MRI tests in future is calculated by multiplying the predicted number of outpatients in future by the number of CT/MRI tests per outpatient number included in the pattern data.

16. The apparatus of claim 1, wherein the predicted number of outpatients in future includes a predicted value for a plurality of future dates.

17. The apparatus of claim 1, wherein the predicted number of outpatients in future is calculated for a plurality of departments.

18. The apparatus of claim 1, wherein the predicted number of outpatients in future is calculated for a plurality of doctors.

19. The apparatus of claim 1, wherein the work schedule comprises a work timetable of each staff member and a number of staff members assigned to each examination room.

20. The apparatus of claim 1, wherein the instructions further cause the processor to adjust the number of staff members to correspond to a minimum value or more assigned in each unit time.
